(19) United States
(12) Patent Application Publication
(10) Pub. No.: US 2009/0082437 A1
(43) Pub. Date: Mar. 26, 2009

(54) Use of Oxaliplatin for Enhancing Radiosensitivity in Radiotherapy of Cervical Cancer

(75) Inventor: Yu-Jen Chen, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: MACKAY MEMORIAL HOSPITAL, Taipei City (TW)

(21) Appl. No.: 11/861,831

(22) Filed: Sep. 26, 2007

(51) Int. Cl. A61K 31/282; A61P 35/00

(52) U.S. Cl. 514/492

(57) ABSTRACT

The present invention relates to a method for enhancing radiosensitivity in radiotherapy of cervical cancer by administering to cervical cancer cells an effective amount of oxaliplatin. Radiation combined with pretreatment of oxaliplatin according to the present invention helps to enhance the cytotoxicity of radiation and result in augmenting radiation-induced mitotic catastrophe. The analysis on molecular mechanism revealed that oxaliplatin augmented the radiation-induced DNA double-strand break indicated by reducing gamma-H2AX expression, abrogated radiation-induced ATM phosphorylation and reduced the Chk2 phosphorylation. Oxaliplatin can be used as a promising radiosensitizer for treatment of cervical cancer in radiotherapy.

### BACKGROUND OF THE INVENTION

1. Field of the Invention

The present invention relates to methods for enhancing the effectiveness of cancer therapies. More specifically, the invention relates to methods for enhancing radiosensitivity in radiotherapy of cervical cancer with oxaliplatin.

2. The Prior Arts

Invasive carcinoma of the uterine cervix remains the most common invasive cancer in women worldwide. Cervical cancer is by now still keeping its high rank incidence as one of the most popular cancers of women in many countries. Concurrent chemoradiotherapy (CCRT), combing simultaneously chemotherapy with radiotherapy (RT), is now recommended as a standard treatment for locally advanced and high-risk cervical carcinoma. Radiosensitizers are agents used to overcome the radio-resistant clones, improve the efficiency of radiation, and therefore, reduce the normal tissue complication probability in CCRT by sensitizing tumor cells to radiation. One of the radiosensitizing mechanisms of typical radiosensitizers is increasing DNA damage or inhibiting DNA repair system and DNA synthesis in cancer cells during radiotherapy. Although the local control rate and survival have improved with use of CCRT, the treatment does cause greater toxicity in the bone marrow, gastrointestinal system and the other tissues compared with that caused by radiotherapy alone. The most widely used chemotherapeutic agent as radiosenstitizer for CCRT is cisplatin with extensive clinical evidence. However, the renal toxicity of cisplatin and drug resistance to cisplatin remain major concerns in clinical practice. (Ferrandina, G., Lauriola, L., Distefano, M. G., Zannoni, G. F., Gessi, M., Legge, F., Maggiano, N., Mancuso, S., Capelli, A., Scambia, G. and Ranelletti, F. O. 2002. Increased cyclooxygenase-2 expression is associated with chemotherapy resistance and poor survival in cervical cancer patients. J. Clin. Oncol. 20(4):973-981) Given these limitations, it is desirable to develop and look for novel radiosenstitizers that augment the efficacy of RT for cervical cancer, thus allowing a lower RT dose, and have acceptably low toxicity to improve the current practice.

Oxaliplatin is a platinum derivative with 1,2-diaminocyclohexane (DACH) carrier ligand. It is a novel chemotherapeutic agent effective against advanced colorectal cancer. Unlike other platinum-based agents, oxaliplatin does not induce dose-limiting nephrotoxicity and dose limiting bone marrow toxicity is uncommon. DACH-platinum adducts formed by oxaliplatin are bulkier and have been reported more effective at inhibiting DNA synthesis than cis-diammine-platinum adducts formed by cisplatin and carboplatin. Platinum derivatives containing DACH carrier ligand possessed antitumor activity in cells acquired cisplatin resistance. (Mamenta, E. L., Poma, E. E., Kaufmann, W. K., Delmastro, D. A., Grady, H. L. and Chaney, S. G. 1994. Enhanced replicative bypass of platinum-DNA adducts in cisplatin-resistant human ovarian carcinoma cell lines. Cancer Res. 54(13):3500-3505.) These derivatives, including oxaliplatin, have less nephrotoxicity of cisplatin and less myelosuppression of carboplatin.

Taken together, given these limitations of CCRT in cervical cancer, it is desirable to develop methods for enhancing radiosensitivity and look for novel radiosenstitizers that augment the efficacy of RT for cervical cancer, thus allowing a lower RT dose, and have acceptably low toxicity to improve the current practice. Accordingly, it would be advantageous to evaluating the effect and possible mechanism of oxaliplatin on enhancing the radiosensitivity in human cervical cancer. And it will greatly contribute beneficial effects on cancer treatment if oxaliplatin, having less nephrotoxicity and bone marrow toxicity, can be applied in the radiation treatment of cervical cancer.

### SUMMARY OF THE INVENTION

The objective of the present invention is to provide a method for enhancing radiosensitivity of cervical cancer. Especially, the method relates to administering effective amount of oxaliplatin to a cervical cancer cell before ionizing radiation, and identify if oxaliplatin can sensitize the cervical cancer cell in radiation therapy for applying oxaliplatin as a radiosensitizer in cervical cancer treatment.

To fulfill the abovementioned objectives, the present invention evaluates the effectiveness of oxaliplatin on enhancing the radiosensitivity of cervical cancer cell lines through colony formation assay, cell cycle distribution analysis, and determines possible mechanisms by cell morphology and DNA damage repair analysis. Human cervical cancer cell lines HeLa and SiHa were irradiated with or without oxaliplatin pretreatment. The surviving fraction and sensitizer enhancement ratio (SER) were estimated by using a colony formation assay and linear-quadratic model. The cell-cycle distribution analysis was evaluated by using propidium iodide staining and flow cytometry. Cell morphology was observed after staining with Wright's dye. For assessing DNA damage repair machinery, the cellular protein was subjected to Western blotting and immunofluorescence for observing the expression of DNA damage-related molecules.

The non-toxic dose of oxaliplatin to HeLa and SiHa cells was 5 μM and 10 μM, respectively. The colony formation assay showed pretreatment with oxaliplatin at 5 μM to 10 μM markedly decreased the survival of irradiated tumor cells. The maximal SERs at 37% survival were 3.4 in HeLa and 4.8 in SiHa cells. Furthermore, the results of cell morphology demonstrate that oxaliplatin pretreatment led to enhance the morphological changes characteristic of mitotic catastrophe which was induced by ionizing radiation. The DNA damage repair analysis represented oxaliplatin modulated the repair of radiation-induced DNA double-strand breaks indicated by reducing the initial intensity of gamma-H2AX foci, abrogated radiation-induced ataxia telangiectasia-mutated (ATM) phosphorylation and reduced the checkpoint kinase 2 (Chk2) phosphorylation.

According to above results of the present invention, oxaliplatin appears to be a promising radiosensitizer for enhancing radiosensitivity of human cervical cancer cells, further to use against human cervical cancer and obtain better effects in combination with radiation therapy via modulating ATM and Chk2 activation during DNA damage repairing.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

The related drawings in connection with the detailed description of the present invention to be made later are described briefly as follows, in which:

FIG. 1 is survival curves of cervical cancer cells treating without or with various dose (1.25, 2.5, 5, 10 μM) oxaliplatin before radiation. (A) HeLa cell line; (B) SiHa cell line. Data points and vertical bars represent the mean and standard errors of three independent experiments.

FIG. 2 is DNA histograms in cell cycle distribution of HeLa cells treating without or with various dose (1.25, 2.5, 5, 10 μM) oxaliplatin before radiation by flow cytometry.

FIG. 3 is morphology of cervical cancer HeLa cells treated without or with various dose (1.25, 2.5, 5, 10 μM) oxaliplatin before radiation. (A) untreated control; (B) treating with 5 μM oxaliplatin for 2 h; (C) treating with radiation 2 Gy alone; (D) treating with 5 μM oxaliplatin for 2 h followed by radiation 2 Gy. All pictures are at the same magnification (1000×). Bar, 20 μm. The arrows indicate representative cell with typical characteristics of mitotic catastrophe.

FIG. 4 is immunofluorescent staining for expression of gamma-H2AX in cervical cancer HeLa cells. (A) untreated control; (B) treating with 5 μM oxaliplatin for 2 h; (C) treating with radiation 2 Gy alone; (D) treating with 5 μM oxaliplatin for 2 h followed by radiation 2 Gy. Red color represented the result of staining with Rhodamine-conjugated gamma-H2AX antibody. Blue color represented the result of counterstaining with DAPI. All pictures are at the same magnification (400×).

FIG. 5 is Western blot analysis for expression levels of molecules involving DNA damage repair: pATM, pChk1Ser³¹⁷, pChk2Thr⁶⁸, pAkt, and Rad51 in HeLa cells treating with various conditions. Actin served as an internal loading control.

### DETAILED DESCRIPTION OF THE PREFERRED EMBODIMENT

The present invention evaluated the effectiveness and possible mechanism of oxaliplatin combined with radiation in enhancing radiosensitivity of human cervical cancer cells. From the results and observation of colony formation assay, cell cycle analysis, morphologies, and DNA damage repair analysis, there were significant differences in radiation survival between cervical cancer cells treated with or without oxaliplatin and proved that pretreatment with oxaliplatin can enhance the cytotoxicity of radiation. In addition, oxaliplatin pretreatment resulted in moderate arrest at G2/M phase and augmented radiation-induced mitotic catastrophe, one type of cell death, in cervical cancer cells. The analysis on molecular mechanism revealed that oxaliplatin augmented the radiation-induced DNA double-strand break indicated by reducing initial gamma-H2AX expression, abrogated radiation-induced ataxia telangiectasia-mutated (ATM) phosphorylation and reduced the checkpoint kinase 2 (Chk2) phosphorylation. According to the present invention, oxaliplatin can be used as a radiosensitizer for treatment of cervical cancer in radiotherapy. The details of the corresponding test methods to evaluate radiosensitivity effect of oxaliplatin are described in examples as follows.

### Example 1

### Effect of Oxaliplatin Treatment on Viability of Cervical Cancer Cells

An ideal radiosensitizer means it enhances radiosensitivity at non-toxic dose, but not due to the combined toxicity of drug and radiation. Thus to determine the toxic effect of different oxaliplatin concentrations on human cervical cancer cells by using 3-(4,5-dimethylthiazol-2-yl)-2, S-diphenyl tetrazolium bromide (MTT) assay. This assay is commonly used to determine cell proliferation, percent of viable cells, and cytotoxicity. MTT is a yellow dye, which can be absorbed by the living cells and be reduced to purplish blue formazan crystals by succinate tetrazolium reductase in mitochondria. Formazan formation can therefore be used to assess and determine the survival rate of cells.

In preliminary work, the human cervical cancer cell lines HeLa (HPV 18+) and SiHa (HPV 16+) were obtained from American Type Culture Collection (Manassas, Va.). These cells were cultured as monolayers and maintained in DMEM (GIBCO, Grand Island, N.Y., USA) medium supplemented with 100 units/mL penicillin (Invitrogen, Carlsbad, Calif.), 100 μg/mL streptomycin (Invitrogen, Carlsbad, Calif.), 2 mM-glutamine (Invitrogen, Carlsbad, Calif.), and 10% fetal bovine serum (Atlanta Biologicals, Norcross, Ga.) at 37° C. in a humidified 5% CO₂ incubator. On the other hand, oxaliplatin was purchased from TTY Biopharm Co. Ltd (Taiwan). The powder was dissolved in PBS to produce an aqueous stock solution and diluted with PBS for future use.

The human cervical cancer cell lines HeLa (HPV 18+) and SiHa (HPV 16+) were separately cultivated in media containing various doses (0˜10 μM) of oxaliplatin for 24 hours. Then resuspended the cells in fresh medium and cultured for 2 days before MTT assay. For the assay, the cells were incubated with the MTT (tetrazolium compounds) for 4 h, lysed with DMSO, and the color crystals were solubilized with an ELISA reader at a wavelength of 570 nm.

From the results of cells viability, after 24 hours' exposure, the highest non-toxic (cell viability>90%) dose of oxaliplatin for HeLa and SiHa cells was 5 μM and 10 μM, respectively. (data not shown)

### Example 2

### Effect of Oxaliplatin Pretreatment on Radiosensitivity of Cervical Cancer Cells

To evaluate whether oxaliplatin treatment influenced the radiosensitivity of cervical cancer cells, this experiment treated HeLa and SiHa cells with oxaliplatin at various concentrations for 2 h prior to radiation survival assays in the invention.

Based on the above results of cell viability assessment, 0˜10 μM oxaliplatin was used to test for radiation sensitization. HeLa and SiHa were pretreated with various doses (0, 1.25, 5, and 10 μM) of oxaliplatin for 2 h, and the oxaliplatin was washed out before radiation delivery. Irradiation was performed with 6 MeV of electron beam by a linear accelerator (Clinac® 1800, Varian Associates, Inc.) with various doses (0.5, 1, 2 and 3 Gy) at a dose rate of 2.4 Gy/min in a single fraction. Full electron equilibrium was ensured for each fraction by a parallel plate PR-60C ionization chamber (CAPINTEL, Inc., Ramsey, N.J.). Following radiation, a colony formation assay was performed. The ionizing radition of the present invention used include, but not limited to, electron beam radition, photon, electron, gamma, alpha, beta, neutron, and proton radiation. Among them, electron beam radition is preferred.

Viable tumor cells (10³) were plated onto culture dishes (35 mm²) and allowed to grow in McCoy's 5A medium containing 20% heat-inactivated FCS and 0.24% agarose at 37° C. in a humidified 5% CO₂ incubator. After 10 to 14 days, the resultant colonies were stained with 0.4% crystal violet and the colonies containing at least 50 cells were determined. The surviving fraction was calculated as the mean colonies/(cells inoculated×plating efficiency). The control plating efficiency for HeLa and SiHa cells was 40-60%. Survival curves were fitted by a linear-quadratic model. The sensitizer enhancement ratio (SER) was calculated as the radiation dose needed for radiation alone divided by the dose needed for oxaliplatin plus radiation to yield a surviving fraction of 37% (Do in radiobiology).

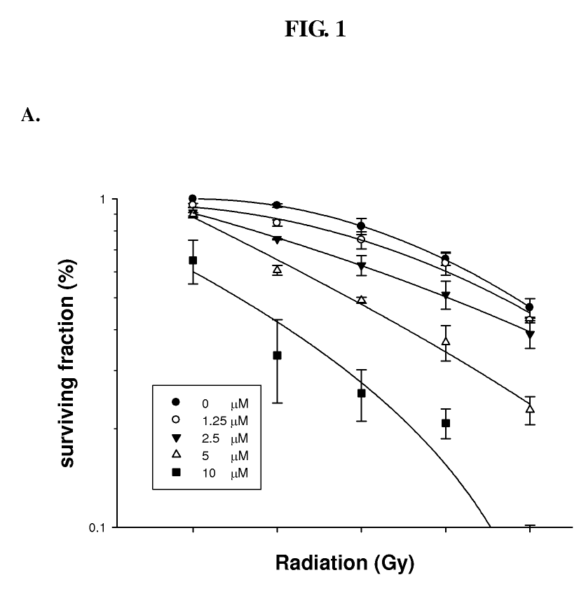

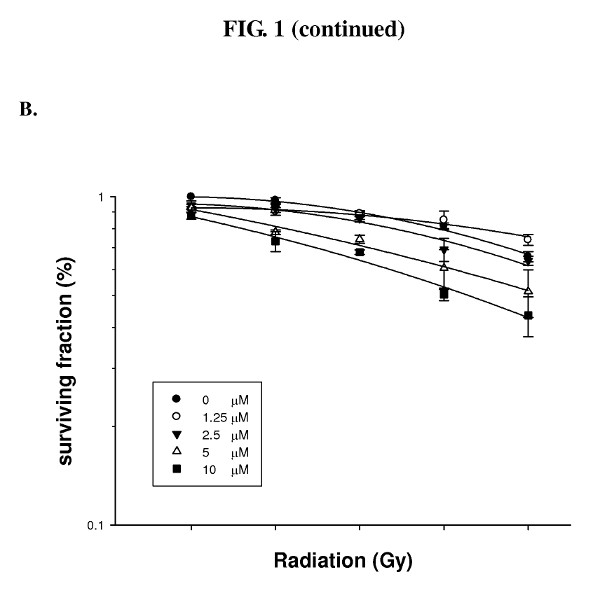

Statistical analysis was performed by analysis of variance when comparing the colony formation between different treatment conditions. All data were presented as mean ±standard error of the mean (SEM). SPSS 10.0 software (SPSS Inc, Chicago) was used for analysis of all the data. Statistical significance was assumed where the p value was less than 0.05. The result is shown in FIG. 1.

Refers to FIG. 1, the radiation survival curves of cervical cancer cells pre-treating with various dose of oxaliplatin combined with radiation. Significant difference were observed in survival curves between treatments with radiation alone and radiation plus oxaliplatin in the radiation dose range of 0.5-3 Gy. Oxaliplatin at low dose range sensitized HeLa and SiHa cells to radiation in a dose-dependent manner and enhanced the cytotoxicity of radiation. The radiation of untreated HeLa cells at a dose of 0-3 Gy reduced the surviving fraction down to 46.6%. Pretreatment with oxaliplatin at 5 and 10 μM markedly decreased the survival of irradiated tumor cells compared to those treated only with radiation (seen in FIG. 1A and FIG. 1B). The calculated SERs of oxaliplatin on HeLa cells were 1.7 and 3.4 for doses of 5 and 10 μM, respectively. Oxaliplatin had a similar effect on SiHa cells with a maximal SER 4.8.

In addition, large portions of the colorectum, small intestine, and urinary bladder and the normal tissues adjacent to them are inevitably included within the radiation field and limit the demand dose in clinical radiotherapy of cervical cancer. In the present invention, oxaliplatin can sensitize cervical cancer cell lines and enhance the radiation efficiency to 3.1-3.9 times than radiation alone. This represented the using of oxaliplatin as a radiosensitizer can reduce required radiation dose and prevent radiation injury to normal tissues.

On the other hand, several lines of evidence indicate that high-risk HPV (human papillomaviruses) infection is an important event in the progression of cervical cancer. The cervical cancer cell lines HeLa and SiHa in the present invention are the most frequent using type cell lines in current clinical study. HeLa cell is a HPV 18 positive adenocarcinoma cell and SiHa cell is a HPV 16 positive squamous cell carcinoma. Studies have revealed cisplatin combined with radiation has no effect on HPV 18 positive adenocarcinoma cell (HeLa). Therefore oxaliplatin, has less nephrotoxicity and bone marrow toxicity than cisplatin, can be applied to combine radiation for treatment of HPV 18 positive type cervical cancer cell (HeLa) instead of cisplatin.

### Example 3

### Effect of Oxaliplatin on Cell Cycle Distribution

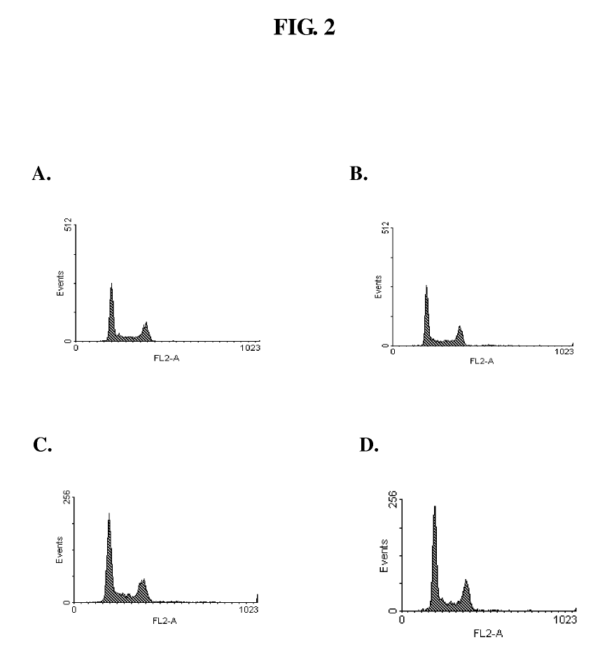

To eliminate the potential contribution of cell cycle redistribution to the observed radiosensitization, flow cytometric analysis was performed to determine cell cycle progression after treatment of oxaliplatin. After treating cervical cancer cell line HeLa with 5 μM oxaliplatin for 2 h or 24 h after radiaton, drugs was washed out and cells were cultured for further 24 h. HeLa cells were harvested and fixed with 70% ethanol at 4° C. for 1 h. The cells were stained for 30 min with propidium iodide solution (containing propidium iodide, 0.5 mg/mL; RNAse, 0.1 mg/mL) from a CycleTEST PLUS DNA reagent kit (Becton Dickinson, Lincoln Park, N.J.). Analysis of the DNA content was performed using a FACScaliber flow cytometer (Becton Dickinson). The data from 10⁴ cells were collected and analyzed using ModFit software (Becton Dickinson). The results are shown in FIG. 2.

The effect of oxaliplatin on cell cycle distribution was evaluated under the same conditions affecting radiosensitization by DNA histograms shown in FIG. 2. Compared with untreated exponentially growing HeLa cells by software, after 2 hours' treatment with oxaliplatin (5 μM), the proportion in the G0/G1 phase was decreased, whereas the percentage in the G₂/M phase was increased. (seen in FIG. 2B) It suggested that the cells were arrested in the G2/M phase of the cell cycle. The proportion of cells in the S phase remained unchanged after treatment with oxaliplatin. Radiation alone affected cell cycle distribution with an increasing number of cells in the in G₂/M phase. (seen in FIG. 2C) Pretreatment with oxaliplatin further increased the number. (seen in FIG. 2D) The percentage of cells at G2/M phase were 22.2%±1.0, 25.1%±2.1, 26.2%±3.7, and 32.3%±3.5 for cells untreated and those treated with oxaliplatin, irradiation, and oxaliplatin plus irradiation, respectively.

Generally, cells in the mitotic phase (M) are most radiosensitive, followed by G2 cells, and then early G1 cells. Thus to arrest cell progression in G2/M is one strategy to sensitize tumor cells to radiation. In this invention, oxaliplatin pretreatment resulted in augmenting the radiation-induced cell cycle accumulation in G2/M phase, demonstrating that oxaliplatin is a potent radiosensitizer in fractionated radiotherapy of cervical cancer.

### Example 4

### Effect of Oxaliplatin on Morphology of Cervical Cancer Cells

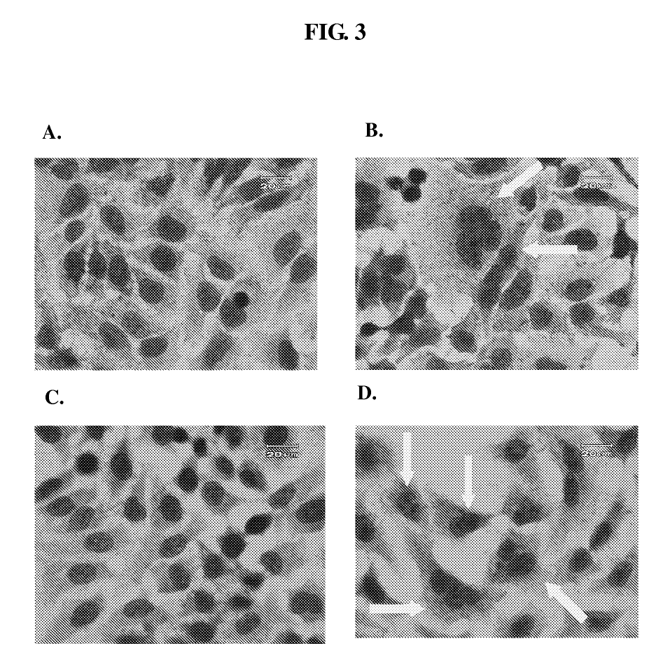

After seeding for 24 h, HeLa cells were treated with various conditions including untreated control, oxaliplatin 5 μM for 2 h, radiation 2 Gy, and oxaliplatin 5 μM for 2 h followed by washing out and radiation 2 Gy. After these treatments, cell were cultured for further 24 h and then collected for Wright's staining and observation under a light microscope. The results are summarized in FIG. 3.

As the results shown in FIG. 3, the percentage cells with mitotic catastrophe in untreated control, oxaliplatin, radiation and oxaliplatin plus radiation was 5.6±1.3%, 27.3±2.2%, 16.7±3.2% and 38.5±3.8%, respectively. The arrows indicate representative cell with typical characteristics of mitotic catastrophe. Compared to the results of other treatment conditions, oxaliplatin pretreatment enhanced the morphological changes characteristic of mitotic catastrophe which was induced by ionizing radiation. In additional, no marked changes were seen in the percentages of apoptosis and necrosis between all groups.

### Example 5

### Effect of Oxaliplatin on Regulation of DNA Damage Repair Machinery

To understand the mechanism of action, DNA damage repair machinery was examined due to the ability of oxaliplatin to form DNA adducts by using immunofluorescent staining and Western blotting.

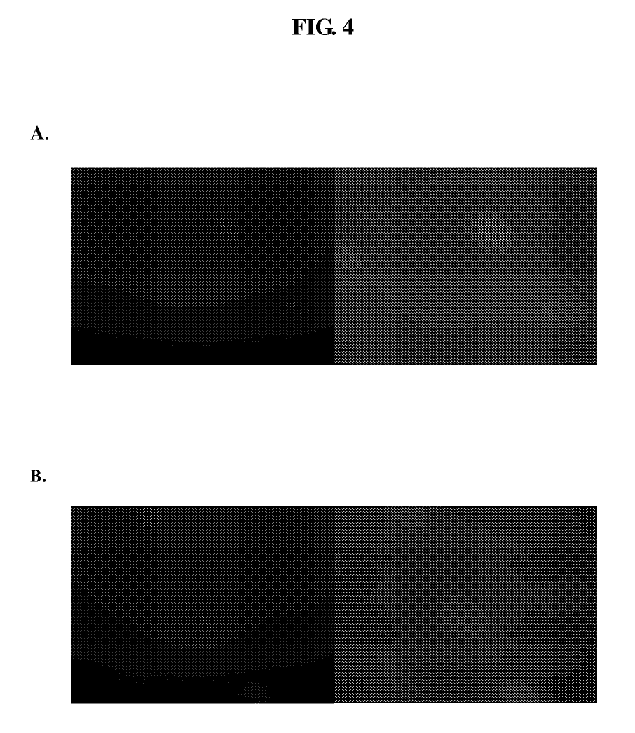

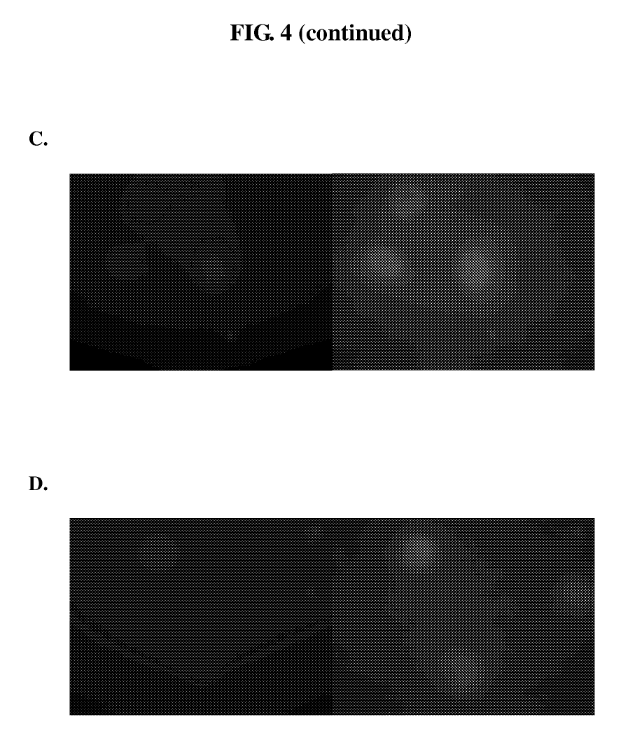

Immunofluorescent staining was used to determine radiation-induced expression of gamma-H2AX which is an indicator of DNA double-strand break. For immunofluorescent staining of gamma-H2AX, HeLa cervical cancer cells that were untreated (control), treated with oxaliplatin 5 μM for 2 h, irradiation of 2 Gy, and oxaliplatin plus irradiation, cells were harvested 15 min after radiation, stained with Rhodamine-conjugated gamma-H2AX antibody (red) and counterstained with DAPI (blue), respectively. The results are shown in FIG. 4.

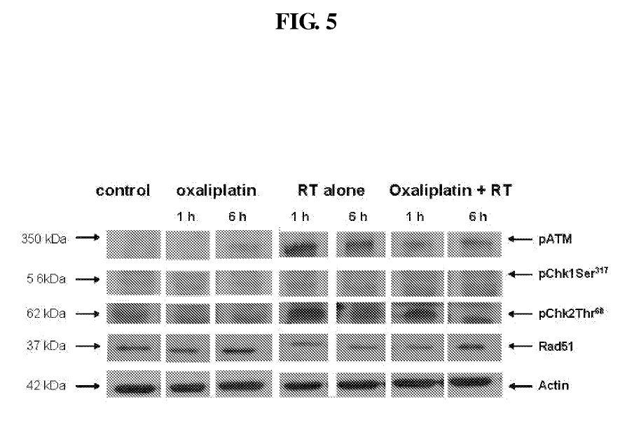

For western blot analysis, HeLa cells were cultured with various treatments: untreated (control), oxaliplatin 5 μM for 2 h (oxaliplatin), radiation 2 Gy (RT alone) and oxaliplatin plus radiation (oxaliplatin+RT) for further 1 h and 6 h. Then, the cellular proteins were extracted, quantified and subjected to gel electrophoresis using 10% (wt/vol) SDS-polyacrylamide gels. Protein samples were then blotted onto polyvinylidene difluoride (PVDF) membranes (Bio-Rad, Hercules, Calif.). The membranes were probed with diluted primary antibodies and detected using horseradish peroxidase-conjugated anti-mouse IgG followed by the use of enhanced chemiluminescence kits (Amersham Pharmacia Biotech). Anti-actin antibody was used as an internal control. The results are shown in FIG. 5.

Various biological stresses activate cell cycle checkpoints, which halt cell cycle progression and allow for either DNA repair or apoptotic pathways to function. Ataxia-Telangiectasia mutated (ATM) is one kind of protein kinases important for cell cycle checkpoint induction after DNA damage. ATM principally phosphorylate and activate checkpoint kinase 1 (Chk1) and checkpoint kinase 2 (Chk2) which are effector kinases in the cellular DNA damage response and impairment of their function is closely related to tumorigenesis. The phosphorylation of H2AX, a substrate of ATM, to be gamma-H2AX has been used as an indicator of DNA DSB, a marker for estimation of DNA repair and a target of radiosensitization. The result of immunofluorescent staining shown in FIG. 4 demonstrated that pretreatment with oxaliplatin reduced radiation-induced early expression of gamma-H2AX, indicating a correlation between modulation of ATM-Chk2 axis and repair of DNA DSB.

FIG. 5 shows the Western blot analysis for molecules involving DNA damage repair. Ionizing radiation (RT alone) induced extensive expression of phosphorylated ATM and 5 μM oxaliplatin for 2 h pretreatment plus radiation (oxaliplatin+RT) markedly reduced this induction. Phosphorylation of Chk2, the downstream DNA damage repair molecule, was augmented by ionizing radiation (RT alone) and oxaliplatin pretreatment plus radiation (oxaliplatin+RT) reduced it. In addition, no effect on expression of rad51 and Chk1 phosphorylation in irradiated cells was noted by oxaliplatin.

Above results showed that oxaliplatin inhibited radiation-induced phosphorylation of ATM and Chk2, but not Chk1, in cervical cancer HeLa cells. Oxaliplatin can also reduce gamma-H2AX expression. Taken together, it suggests that oxaliplatin might sensitize HeLa cells to ionizing radiation through modulating ATM-Chk2 axis in inhibiting the repair of DNA double-strand breaks (DSB). In addition, oxaliplatin inhibited Chk2 activation and enhanced the radiation-induced mitotic catastrophe in HeLa cells. Given that mitotic catastrophe is the predominant type of radiation-induced cell death in several kinds of cancer cells, it indicates that oxaliplatin may promote development of mitotic catastrophe with involvement of Chk2 inhibition in HeLa cells.

In conclusion, oxaliplatin had a radiosensization effect on human cervical cancer HeLa and SiHa cells to ionizing radiation. This effect may involve the modulation of radiation-induced ATM and Chk2 activation during repair of DNA double-strand breaks. Combined with the apparent benign effect on normal cells, oxaliplatin may render it a useful adjuvant to radiotherapy for cervical cancer.

What is claimed is:

1. A method for enhancing radiosensitivity of a cervical cancer cell, comprising:
administering to the cervical cancer cell an effective radiosensitizing dose of oxaliplatin.

2. The method as claimed in claim 1, wherein the effective dose of oxaliplatin is from about 1.25 μM to about 10 μM.

3. The method as claimed in claim 1, wherein the cervical cancer cell is form HeLa or SiHa cell line.

4. The method as claimed in claim 3, wherein an effective non-toxic dose of oxaliplatin administered to the HeLa cell line is 5 μM.

5. The method as claimed in claim 3, wherein an effective non-toxic dose of oxaliplatin administered to the SiHa cell line is 10 μM.

6. The method as claimed in claim 1, wherein oxaliplatin is administered before ionizing radiation.

7. The method as claimed in claim 6, wherein the ionizing radition is photon or electron, gamma, alpha, beta, neutron, or proton radiation.

8. The method as claimed in claim 7, wherein the ionizing radition is electron beam radiation.

9. The method as claimed in claim 8, wherein a proper dose of the electron beam radiation is from about 0.5 Gy to about 3 Gy.

10. The method for augmenting the cytotoxicity of radiation and radiation-induced mitotic catastrophe in a cervical cancer cell, comprising:
administering to the cervical cancer cell an effective radiosensitizing dose of oxaliplatin; and
exposing the cervical cancer cell to ionizing radiation.

11. The method as claimed in claim 10, wherein the effective dose of oxaliplatin is from about 1.25 μM to about 10 μM.

12. The method as claimed in claim 10, wherein a proper dose of the ionizing radiation is from about 0.5 Gy to about 3 Gy.

13. The method for augmenting radiation-induced DNA double-strand break in a cervical cancer cell, comprising:
administering to the cervical cancer cell an effective radiosensitizing dose of oxaliplatin; and
exposing the cervical cancer cell to ionizing radiation.

14. The method as claimed in claim 13, wherein the augment of DNA double-strand break is caused by inhibiting phosphorylation of Ataxia-Telangiectasia mutated (ATM) and checkpoint kinase 2 (Chk2).

15. The method as claimed in claim 13, wherein the augment of DNA double-strand break is caused by reducing the amount of gamma-H2AX expression.

16. The method as claimed in claim 13, wherein the effective dose of oxaliplatin is from about 1.25 μM to about 10 μM.

17. The method as claimed in claim 13, wherein a proper dose of the ionizing radiation is from about 0.5 Gy to about 3 Gy.
